SEQUENCE LISTING (12 sequences)

SEQ ID NO: 1 - DNA, 1593 bp, synthetic construct
  source 1..1593: mol_type = other DNA
atgatgatggcttccaaggatgctacctcctccgttgacggagcttccggtgctggtcaattggttccagaagttaacgcttccgacccattggctatggatccagttgctggttcttctactgctgttgctactgctggtcaagttaatccaattgatccttggattattaacaactttgttcaagctccacaaggtgaatttactatttctccaaacaacactccaggtgatgttttgttcgatttgtctttgggtccacatttgaacccatttttgttgcatttgtcccaaatgtataacggttgggttggtaacatgagagttagaattatgttggctggtaatgcttttactgctggtaagattattgtttcttgtattccacctggttttggttctcataacttgaccattgctcaggctactttgtttcctcatgttattgctgatgttagaactttggatccaattgaagttccattggaagatgttagaaacgttttgtttcataacaacgacagaaaccaacaaactatgagattggtttgtatgttgtataccccattgagaactggaggtggtactggtgattcttttgttgttgctggtagagttatgacttgtccttctcctgattttaactttttgtttttggttcctccaactgttgagcagaagactagaccatttactttgccaaatttgccattgtcttctttgtctaattccagagctccattgccaatttcttctatgggtatttctcctgataacgttcaatccgttcaatttcaaaacggtagatgtaccttggatggtagattggttggtactactcctgtttctttgtctcatgttgctaagattagaggtacttccaacggtactgttattaacttgactgaattggatggtactccatttcatccttttgagggtcctgctccaatcggatttcctgatttgggtggttgtgattggcatattaatatgactcagtttggtcattcttctcagactcaatacgatgttgacactactccagatacttttgttcctcatttgggttctattcaagctaacggtatcggttctggtaactacgttggtgttttgtcttggatttctcctccttctcacccatctggttctcaagttgacttgtggaagattccaaactacggttcttccattactgaagctactcatttggctccttctgtttacccacctggtttcggtgaagttttggttttttttatgtctaagatgccaggtcctggtgcttacaacttgccatgtttgttgcctcaagaatatatttctcatttggcttctgagcaggctcctactgttggtgaagctgctttgttgcattacgttgatccagatactggtagaaatttgggagaatttaaggcttacccagatggttttttgacttgtgttccaaacggtgcttcttctggtcctcaacaattgccaattaacggtgttttcgtttttgtttcctgggtttctagattttatcagttgaagcctgttggtactgcttcttctgctagaggtagattgggtttgagaagataa

SEQ ID NO: 2 - protein, 530 aa, synthetic construct
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 3 - DNA, 1629 bp, synthetic construct
  source 1..1629: mol_type = other DNA
atgaagatggcttccaacgacgctgctccatctaccgacggtgctgctggtttggttccagaatctaacaacgaagttatggctttggaacctgttgctggtgctgctttggctgctccagttaccggtcagactaatattattgatccatggattagagctaactttgttcaagctccaaacggagaatttactgtttctcctagaaacgctcctggtgaagttttgttgaatttggagttgggtccagaattgaacccatacttggctcacttggctagaatgtataacggttacgctggtggtatggaagttcaagttatgttggctggtaatgcttttactgctggtaagttggtttttgctgctgttcctcctcactttccagttgagaatttgtctcctcaacaaattactatgttcccacatgttattatcgacgttagaactttggaacctgttttgttgcctttgcctgatgttagaaataacttttttcactacaaccagaaggatgatccaaagatgagaattgttgctatgttgtacaccccattgagatccaacggttctggtgatgatgtttttactgtttcctgtagagttttgactagaccatctccagattttgattttacttacttggttcctccaactgttgagtccaaaaccaagccatttactttgcctattttgactttgggtgaattgtctaactccagatttccagtttctattgatcaaatgtacacttctccaaacgagattatttctgttcaatgtcaaaacggtagatgtactttggatggagaattgcagggtactactcaattgcaagtttctggtatttgtgcttttaagggtgaagttactgctcatttgcatgataacgatcatttgtacaacgttactattactaacttgaacggttctccatttgatccatctgaggatattcctgctccattgggtgttccagattttcagggtagagtttttggtattatttctcaaagagacaagcataactccccaggtcataacgaaccagctaacagaggtcatgatgctgttgttccaacctacactgctcaatacactccaaagttgggtcagattcaaattggtacatggcaaaccgatgatttgactgttaaccaaccagttaaattcacaccagttggtttgaatgataccgaacattttaatcaatgggttgttcctagatatgctggtgctttgaatttgaatactaacttggctccttccgttgctccagtttttccaggtgaaagattgttgttttttagatcttacatccctttgaagggtggttatggtaacccagctattgattgtttgttgccacaggagtgggttcaacatttttaccaagaggctgctccatcaatgtctgaggttgctttggttagatatattaacccagatactggtagagctttgtttgaggctaagttgcatagagctggttttatgacagtttcttctaacacttctgctccagttgttgttccagctaatggttattttagatttgattcttgggttaaccagttttattctttggctccaatgggtactggtaatggtagaagaagagttcaataa

SEQ ID NO: 4 - protein, 542 aa, synthetic construct
MKMASNDAAPSTDGAAGLVPESNNEVMALEPVAGAALAAPVTGQTNIIDPWIRANFVQAPNGEFTVSPRNAPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPTVESKTKPFTLPILTLGELSNSRFPVSIDQMYTSPNEIISVQCQNGRCTLDGELQGTTQLQVSGICAFKGEVTAHLHDNDHLYNVTITNLNGSPFDPSEDIPAPLGVPDFQGRVFGIISQRDKHNSPGHNEPANRGHDAVVPTYTAQYTPKLGQIQIGTWQTDDLTVNQPVKFTPVGLNDTEHFNQWVVPRYAGALNLNTNLAPSVAPVFPGERLLFFRSYIPLKGGYGNPAIDCLLPQEWVQHFYQEAAPSMSEVALVRYINPDTGRALFEAKLHRAGFMTVSSNTSAPVVVPANGYFRFDSWVNQFYSLAPMGTGNGRRRVQ

SEQ ID NO: 5 - DNA, 1647 bp, synthetic construct
  source 1..1647: mol_type = other DNA
atgaagatggcttccaacgacgctactccatctaacgacggtgctgctggtttggttccagaaattaacaacgaagctatggctttggacccagttgctggtgctgctattgctgctccattgaccggtcagcaaaacattattgacccatggattatgaataacttcgttcaagctccaggtggagaatttactgtttcccctagaaactctcctggtgaagttttgttgaacttggagttgggtccagaaatcaacccatacttggctcacttggctagaatgtacaacggttacgctggtggttttgaagttcaagttgttttggctggtaatgcttttactgctggaaagattattttcgctgctattccacctaattttccaattgataatttgtccgctgctcagattactatgtgtcctcatgttattgttgatgttagacagttggaacctgttaatttgccaatgccagatgttagaaacaactttttccattacaaccaaggttctgattctagattgagattggttgctatgttgtacactccattgagagctaacaactctggtgatgacgtttttaccgtttcttgtagagttttgactagaccttctccagaattttcttttaactttttggttcctccaactgttgaatctaagaccaagccatttactttgccaattttgactatttctgagatgtctaactccagatttccagttcctattgattctttgcacacttctccaactgagaacatcgttgttcaatgtcagaacggtagagttacattggatggtgaattgatgggtactacccaattgttgccatctcaaatttgtgcttttagaggtgttttgactagatctacttccagagcttctgatcaagctgatactgctactcctagattgtttaattactactggcatattcagttggataacttgaacggtactccatacgatccagctgaggacattccaggtccattgggtactccagattttagaggtaaagtttttggtgttgcttcccaaagaaaccctgactctactactagagctcatgaagctaaggttgatactactgctggaagatttactccaaagttgggatctttggagatttctaccgaatctggtgattttgatcaaaaccaaccaactagatttactcctgttggtattggtgttgatcatgaagctgattttcagcagtggtctttgcctgattactctggtcaatttacccataacatgaacttggctccagctgttgctcctaattttcctggtgaacaattgttgtttttcagatctcaattgccttcttccggtggtagatctaacggtattttggattgtttggttccacaagaatgggttcaacatttttaccaagaatctgctccagctcaaactcaggttgctttggttagatatgttaaccctgatactggtagagttttgtttgaagctaagttgcataagttgggttttatgactattgctaagaacggtgattctccaattaccgttccaccaaatggttattttagatttgagtcttgggttaatccattttacactttggctccaatgggtactggtaacggtagaagaagagttcaataa

SEQ ID NO: 6 - protein, 548 aa, synthetic construct
MKMASNDATPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKIIFAAIPPNFPIDNLSAAQITMCPHVIVDVRQLEPVNLPMPDVRNNFFHYNQGSDSRLRLVAMLYTPLRANNSGDDVFTVSCRVLTRPSPEFSFNFLVPPTVESKTKPFTLPILTISEMSNSRFPVPIDSLHTSPTENIVVQCQNGRVTLDGELMGTTQLLPSQICAFRGVLTRSTSRASDQADTATPRLFNYYWHIQLDNLNGTPYDPAEDIPGPLGTPDFRGKVFGVASQRNPDSTTRAHEAKVDTTAGRFTPKLGSLEISTESGDFDQNQPTRFTPVGIGVDHEADFQQWSLPDYSGQFTHNMNLAPAVAPNFPGEQLLFFRSQLPSSGGRSNGILDCLVPQEWVQHFYQESAPAQTQVALVRYVNPDTGRVLFEAKLHKLGFMTIAKNGDSPITVPPNGYFRFESWVNPFYTLAPMGTGNGRRRVQ

SEQ ID NO: 7 - DNA, 1623 bp, synthetic construct
  source 1..1623: mol_type = other DNA
atgaagatggcttcctctgacgctaacccatctgacggttccgctgctaacttggttccagaagttaacaacgaagttatggctttggaaccagttgttggtgctgctattgctgctccagttgctggtcaacaaaacgttatcgacccttggattagaaataactttgttcaggctccaggtggtgaatttactgtttctcctagaaacgctccaggtgaaattttgtggtctgctcctttgggtccagatttgaatccttacttgtctcacttggctagaatgtataacggttacgctggtggatttgaagttcaagttattttggctggtaacgcttttactgctggtaaggttatttttgctgctgttccacctaactttccaaccgagggtttgtccccatctcaagttactatgtttccacacattgttgttgatgttagacaattggaacctgttttgattccattgcctgatgttagaaataacttctaccattacaaccaatctaacgatccaactattaagttgattgctatgttgtacactccattgagagctaacaacgctggtgatgatgtttttaccgtttcttgtagagttttgactagaccatctccagactttgattttatctttttggttcctccaactgttgaatccagaaccaagccattttctgttcctgttttgactgttgaggaaatgaccaattctagatttccaattccattggaaaagttgtttactggtccttcttctgcttttgttgttcaacctcaaaacggtagatgtactactgatggtgttttgttgggtactactcagttgtctcctgttaacatttgtacttttagaggtgatgttactcatattactggttctagaaattacactatgaacttggcttctcagaactggaataattacgatccaactgaagaaattccagctccattgggtactccagattttgttggtaagattcaaggtgttttgactcaaactactagaactgatggttctactagaggacataaggctactgtttacaccggttctgctgattttgctcctaagttgggtagagttcaatttgaaaccgatactgatcatgacttcgaagctaaccaaaacactaagtttaccccagttggagttattcaggatggatctactactcatagaaatgaaccacaacaatgggttttgccttcttactctggaagaaacactcctaacgttcacttggctccagctgttgctccaacttttccaggtgaacaattgttgttttttagatccaccatgccaggttgttctggttacccaaatatggatttggattgtttgttgcctcaggaatgggttcaatatttttaccaagaagctgctccagctcaatctgatgttgctttgttgagatttgttaatcctgatactggtagagttttgtttgagtgtaagttgcataagtctggttacgttaccgttgctcatactggtcaacatgatttggttattccaccaaacggttactttagatttgattcttgggttaaccagttttacactttggctcctatgggtaacggtactggtagaagaagagctttgtaa

SEQ ID NO: 8 - protein, 540 aa, synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTPNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAL

SEQ ID NO: 9 - DNA, 1644 bp, synthetic construct
  source 1..1644: mol_type = other DNA
atgaagatggcttccaacgacgctgctccatctaacgacggtgctgctaacttggttccagaagctaccaacgaagttatggctttggaaccagttgttggtgcttccattgctgctccagttgttggacaacaaaacattattgacccatggattagagaaaatttcgttcaagctccacaaggtgaatttactgtttctcctagaaactctccaggtgaaatgttgttgaacttggaattgggtccagaattgaacccatacttgtctcacttgtctagaatgtataacggttacgctggtggtatgcaagttcaagttgttttggctggtaatgcttttactgctggtaagattattttcgctgctgttccaccacattttcctgttgagaacatttctgctgctcagattactatgtgtccacatgttattgttgatgttagacaattggaaccagttttgttgccattgccagatattagaaacagattttttcactacaaccaggaaaacactccaagaatgagattggttgctatgttgtatactccattgagagctaactctggtgaagatgtttttactgtttcctgtagagttttgaccagaccagctccagattttgaatttacttttttggttccacctactgttgaatctaagaccaagccttttactttgccaattttgactttgggtgaattgtccaactccagatttccagctccaattgatatgttgtataccgatcctaacgaagctattgttgttcaaccacaaaacggaagatgtactttggatggtactttgcagggtacaactcaattggttccaactcaaatttgttcctttagaggtactttggtttctcaaacttctagatctgctgattctaccgactctgctcctagagttagatctcacccattgcatgttcaattgaagaatttggatggtacaccttacgatccaactgatgaggttcctgctgttttgggtgctattgattttaaaggaactgtttttggagttgcttcccaaagaaacaccactggtaactccattggtgctactagagctcatgaagttcatattgatactactaatccaagatacactccaaagttgggatctgttttgatgtactctgaatccaacgatttcgatgacggtcagccaaccagatttactccaattggtatgggtgctgatgattggcatcaatgggaattgccagaatattctggtcatttgactttgaacatgaacttggctccagctttggctccagcttttccaggtgagagaattttgttttttagatctgttgttccatccgctggtggttacggttctggtcatattgattgtttgattcctcaggagtgggttcaacatttttaccaagaagctgctccatcccaatctgctgttgctttgattagatatgttaaccctgatactggtagaaacatttttgaagctaagttgcacagagaaggttttattaccgttgctaactccggaaataacccaattgttgttccaccaaatggttactttagatttgaagcttgggttaatcaattctacactttgactccaatgggaactggtcagggtagaagaagagttcaataa

SEQ ID NO: 10 - protein, 547 aa, synthetic construct
MKMASNDAAPSNDGAANLVPEATNEVMALEPVVGASIAAPVVGQQNIIDPWIRENFVQAPQGEFTVSPRNSPGEMLLNLELGPELNPYLSHLSRMYNGYAGGMQVQVVLAGNAFTAGKIIFAAVPPHFPVENISAAQITMCPHVIVDVRQLEPVLLPLPDIRNRFFHYNQENTPRMRLVAMLYTPLRANSGEDVFTVSCRVLTRPAPDFEFTFLVPPTVESKTKPFTLPILTLGELSNSRFPAPIDMLYTDPNEAIVVQPQNGRCTLDGTLQGTTQLVPTQICSFRGTLVSQTSRSADSTDSAPRVRSHPLHVQLKNLDGTPYDPTDEVPAVLGAIDFKGTVFGVASQRNTTGNSIGATRAHEVHIDTTNPRYTPKLGSVLMYSESNDFDDGQPTRFTPIGMGADDWHQWELPEYSGHLTLNMNLAPALAPAFPGERILFFRSVVPSAGGYGSGHIDCLIPQEWVQHFYQEAAPSQSAVALIRYVNPDTGRNIFEAKLHREGFITVANSGNNPIVVPPNGYFRFEAWVNQFYTLTPMGTGQGRRRVQ

SEQ ID NO: 11 - DNA, 1623 bp, synthetic construct
  source 1..1623: mol_type = other DNA
atgaagatggcttccaacgacgctgctccatctaacgacggtgctgctggtttggttccagaaggtaacaacgaaaccttgccattggaacctgttgctggtgctgctatcgctgctccagttactggtcaaaacaatattattgatccttggattagaaccaactttgttcaggctcctaacggagaatttactgtttctcctagaaattctccaggtgaaattttgttgaatttggagttgggtcctgacttgaacccatacttggctcacttgtctagaatgtataacggttacgctggtggtgttgaagttcaggttttgttggctggtaatgcttttactgctggtaagattttgtttgctgctgttccacctaactttcctgttgagtttttgtcccctgctcagattactatgttgccacatttgattgttgatgttagaactttggaacctattatgattccattgcctgatgttagaaacactttctttcattattccaaccaacctaactctagaatgagattggttgctatgttgtacactccattgagatcaaacggttctggtgatgacgtttttactgtttcctgtagagttttgactagaccaactcctgatttcgaatttacttacttggttcctccttctgttgaatccaaaaccaagccattctctttgcctattttgactttgtctgaattgactaattccagatttccagttccaattgattctttgtttaccgctcaaaacaacgttttgcaagttcagtgtcaaaacggtagatgtactttggatggtgagttgcagggtactactcaattgttgccatctggtatttgtgcttttagaggtagagttactgctcagattaatcaaagagatagatggcatatgcaattgcaaaacttgaacggtactacttacgatccaaccgacgatgttccagctccattgggtacaccagattttaagggtgttgtttttggtatggtttcccagagaaatgttggtaacgatgctccaggttccactagagctcaacaagcttgggtttctacttactctccacaatttgttccaaagttgggatctgttaatttgagaatttctgataacgacgatttccaatttcaaccaacaaagttcactccagttggtgttaatgatgatgatgacggtcatccatttagacaatgggaattgccaaactattctggtgagttgactttgaacatgaacttggctccaccagttgctcctaactttccaggtgaacaattgttgttttttagatctttcgttccttgttctggtggttacaaccaaggtattattgattgtttgatcccacaagaatggattcaacacttttatcaagaatctgctccatctcaatctgatgttgctttgattagatatgttaacccagatactggtagaacattgtttgaagctaagttgcacagatcaggttacattaccgttgctcattccggtgattacccattggttgttccagctaatggtcactttagatttgattcctgggttaatcaattctactctttggctcctatgggtacaggtaacggtagaagaagagctcaataa

SEQ ID NO: 12 - protein, 540 aa, synthetic construct
MKMASNDAAPSNDGAAGLVPEGNNETLPLEPVAGAAIAAPVTGQNNIIDPWIRTNFVQAPNGEFTVSPRNSPGEILLNLELGPDLNPYLAHLSRMYNGYAGGVEVQVLLAGNAFTAGKILFAAVPPNFPVEFLSPAQITMLPHLIVDVRTLEPIMIPLPDVRNTFFHYSNQPNSRMRLVAMLYTPLRSNGSGDDVFTVSCRVLTRPTPDFEFTYLVPPSVESKTKPFSLPILTLSELTNSRFPVPIDSLFTAQNNVLQVQCQNGRCTLDGELQGTTQLLPSGICAFRGRVTAQINQRDRWHMQLQNLNGTTYDPTDDVPAPLGTPDFKGVVFGMVSQRNVGNDAPGSTRAQQAWVSTYSPQFVPKLGSVNLRISDNDDFQFQPTKFTPVGVNDDDDGHPFRQWELPNYSGELTLNMNLAPPVAPNFPGEQLLFFRSFVPCSGGYNQGIIDCLIPQEWIQHFYQESAPSQSDVALIRYVNPDTGRTLFEAKLHRSGYITVAHSGDYPLVVPANGHFRFDSWVNQFYSLAPMGTGNGRRRAQ

(19) United States
(12) Patent Application Publication
(10) Pub. No.: US 2025/0025546 A1
(43) Pub. Date: Jan. 23, 2025

(54) HEXAVALENT NOROVIRUS VLP VACCINE AND A PREPARATION METHOD THEREOF

(71) Applicant: Chengdu Kanghua Biological Products Co., Ltd., Chengdu (CN)

(72) Inventors: Hui Liu, Chengdu (CN); Dongming Zhou, Chengdu (CN); Di Xie, Chengdu (CN); Lihui Lv, Chengdu (CN); Man Xing, Chengdu (CN); Jiling Ren, Chengdu (CN); Wenli Hou, Chengdu (CN)

(21) Appl. No.: 18/430,663

(22) Filed: Feb. 2, 2024

Related U.S. Application Data
(60) Provisional application No. 63/514,799, filed on Jul. 21, 2023.

(51) Int. Cl. A61K 39/12; A61K 39/00; A61P 31/14; A61P 37/04

(52) U.S. Cl. CPC A61K 39/12; A61P 31/14; A61P 37/04; A61K 2039/55505; A61K 2039/575; A61K 2039/70

(57) ABSTRACT

Disclosed are a hexavalent norovirus VLP vaccine formulation and a preparation method thereof. The formulation comprises six proteins having amino acid sequences shown in SEQ ID No. 2, SEQ ID No. 4, SEQ ID No. 6, SEQ ID No. 8, SEQ ID No. 10, and SEQ ID No. 12. A Pichia pastoris expression system was used to develop the hexavalent norovirus vaccine. Sequencing, digestion, and exogenous gene expression detection were conducted to identify recombinant vectors and select high-expression strains. VLPs were purified by sucrose density gradient centrifugation and identified by electron microscopy, with the results demonstrating that the obtained candidate hexavalent vaccine VLPs had a purity of over 95% and each VLP was intact in structure and uniform in size, indicating structural integrity and stable performance of the VLPs. The hexavalent VLP vaccine induced long-lasting and high-titer antibody responses and generated specific blocking antibodies and T cell immune responses.

### CROSS-REFERENCE TO RELATED APPLICATIONS

The application claims priority to U.S. Provisional Patent Application 63/514,799 filed Jul. 21, 2023, entitled “A HEXAVALENT NOROVIRUS VLP VACCINE AND A PREPARATION METHOD THEREOF,” the entire disclosure of which is incorporated herein by reference for all purposes.

### FIELD OF INVENTION

The present invention relates to the field of vaccines and, specifically, relates to a hexavalent norovirus vaccine and a preparation method thereof.

### REFERENCE TO SEQUENCE LISTING

This application contains a Sequence Listing which has been submitted electronically via Patent Center and is incorporated by reference in its entirety. Said .xml copy, created on Feb. 1, 2024, is named 110635-1391907-000100US, and 24,493 bytes in size.

### BACKGROUND

Human norovirus (NoV) is a non-enveloped, single-stranded positive-sense RNA virus of the family Caliciviridae. The virus is the major etiological agent of viral acute gastroenteritis, which is highly contagious and characterized by local outbreaks worldwide. The virus is mainly transmitted by the fecal-oral route; it is also transmitted by contact with patients, contaminated food, infectious aerosols containing the virus, etc. Norovirus infection most commonly leads to vomiting and diarrhea, followed by nausea, abdominal pain, headache, fever, chills, muscle aches, etc., and may result in dehydration or even death in severe cases. The morbidity and mortality of norovirus infection are high in the elderly, children, and immunocompromised populations.

Since the first reported norovirus outbreak in 1969, the virus has caused multiple outbreaks worldwide. At present, the virus has become the major etiological agent in causing human viral diarrhea after rotaviruses. Statistics show that ⅕ of diarrhea worldwide is caused by norovirus. Every year in the world, nearly 700 million people are infected with norovirus and 210,000 people die as a result; the direct economic loss caused by norovirus infection is over 4 billion US dollars per year, and the indirect economic loss is about 60 billion US dollars per year. In China, the infection rate of norovirus is high, with as much as 15.6% of children under 5 years of age infected; epidemics or outbreaks occur every year; recent epidemiological surveys have shown that norovirus is also the main cause of viral diarrhea in children. However, currently, there is no specific treatment for acute gastroenteritis caused by norovirus and no corresponding prophylactic vaccine. Routine preventive measures mainly include providing health education for vulnerable populations, improving public awareness of protection, and promoting good personal hygiene practices. Therefore, there is an urgent need to develop vaccines and therapeutic drugs to prevent the spread of the virus.

The research and development of human norovirus vaccines and related drugs have long been hindered by the lack of in vitro culture systems and cost-effective animal models for human norovirus infection. In September 2016, Ettayebi et al. found that human norovirus can replicate and produce infectious particles in differentiated human intestinal cells, and the infections of different genotypes can be differentially enhanced by bile acids. While this discovery has driven the preclinical study of norovirus to some extent, the present in vitro culture system is still unable to cost-effectively and efficiently produce live viruses in large quantities; therefore, it is unlikely to develop norovirus vaccines using traditional strategies of live or attenuated vaccines.

The major capsid protein VP1 of norovirus is immunogenic and capable of inducing a protective immune response, and it can be expressed and self-assembled into virus-like particles (VLPs) in several in vitro expression systems, which has facilitated progress in recombinant norovirus vaccine research and development in recent years. The bivalent norovirus VLP vaccine (GI.1 and GII.4) developed by Takeda (Japan) using an insect cell-baculovirus expression system has progressed to phase 2b clinical trials. The monovalent oral vaccine (GI.1) developed by Vaxart (USA) using a recombinant human adenovirus 5 (AdHu5) vector has completed the phase 1b clinical trial in 2017. However, the genotypes of norovirus targeted by the two vaccines are so limited that they are not expected to provide broad-spectrum protection against norovirus infection after marketing. At present, the bivalent norovirus VLP vaccine developed by China National Biotec Group (CNBG) using a Hansenula yeast expression system and the tetravalent norovirus VLP vaccine developed by the Institut Pasteur of Shanghai using a Pichia pastoris system both have completed the preclinical studies and been approved for clinical trials. Unfortunately, none of the above norovirus vaccines are sufficient for protection against the epidemic strains emerging in recent years.

Noroviruses are classified into seven genotypes based on the amino acid sequences of their VPI and further subdivided into over 30 genetic subtypes. Among the genotypes, GI, GII, and GVI are closely associated with human diseases, wherein GII is the most prevalent, followed by GI. Epidemiological studies have shown that in China, the GII group is dominated by GII.3, GII.4, and GII.17, and the GI group by GI.1. In contrast, it was found by the CDC epidemiological survey that in 2016, 79% of norovirus-associated gastroenteritis outbreaks in China originated from GII.P16/GII.2 infection. According to an epidemiological survey between 2012 to 2017 on acute gastroenteritis associated with GII norovirus in children in Shanghai, GII.6 had the fourth highest incidence (2.7%).

With the increasing number of norovirus epidemics and outbreaks, the associated disease burden is increasing; therefore, the immediate priority is to develop practical and efficient norovirus vaccines in addition to the development of corresponding therapeutic drugs. Given the large number of prevalent norovirus genotypes, the development of multivalent norovirus vaccines has become a crucial trend in this field.

### SUMMARY OF THE INVENTION

The present invention seeks to provide a hexavalent norovirus vaccine to prevent highly pathogenic subtypes of norovirus and a preparation method thereof.

The technical solutions of the present invention are:

A hexavalent norovirus VLP vaccine formulation comprising six antigenic proteins having amino acid sequences shown in SEQ ID No. 2, SEQ ID No. 4, SEQ ID No. 6, SEQ ID No. 8, SEQ ID No. 10, and SEQ ID No. 12.

In some embodiments, the weight ratio of the six antigenic proteins is 1:1:1:1:1:1.

In some embodiments, the hexavalent norovirus VLP vaccine formulation comprises an aluminum adjuvant.

In some embodiments, the aluminum adjuvant is aluminum hydroxide.

In some embodiments, provided herein is a preparation method of a hexavalent norovirus VLP vaccine formulation, comprising the following steps:
(1) Construct recombinant vectors: insert nucleotide sequences shown in SEQ ID No. 1, SEQ ID No. 3, SEQ ID No. 5, SEQ ID No. 7, SEQ ID No. 9, and SEQ ID No. 11 into pPink-hc vectors, respectively;
(2) Transfer the recombinant vectors constructed in step (1) into Pichia pastoris for expression, and collect and purify proteins expressed from SEQ ID No. 1, SEQ ID No. 3, SEQ ID No. 5, SEQ ID No. 7, SEQ ID No. 9, and SEQ ID No. 11;
(3) Mix the six proteins purified in step (2) in proportion and encapsulate the mixture with the aluminum adjuvant to obtain the hexavalent norovirus VLP vaccine.

In some embodiments, the weight ratio of the six antigenic proteins is 1:1:1:1:1:1.

In some embodiments, the aluminum adjuvant is aluminum hydroxide.

In some embodiments, provided herein is a kit for preparing a hexavalent norovirus VLP vaccine formulation, wherein the kit comprises polynucleotides comprising nucleotide sequences shown in SEQ ID No. 1, SEQ ID No. 3, SEQ ID No. 5, SEQ ID No. 7, SEQ ID No. 9, and SEQ ID No. 11, and a pPink-hc vector. In some embodiments, the kit further comprises an aluminum adjuvant. In some embodiments, the aluminum adjuvant is aluminum hydroxide.

Compared with the prior art, the present invention has the following beneficial effects:

By utilizing a Pichia pastoris expression system for hexavalent norovirus vaccine development, and sequencing, digestion, and exogenous gene expression detection for recombinant vector identification and high-expression strain selection, the inventors have successfully obtained correct clones and high-expression vaccine strains. Through VLP purification by sucrose density gradient centrifugation and identification by electron microscopy, the inventors have obtained candidate hexavalent vaccine VLPs that exhibit a purity of over 95%, with each VLP intact in structure and uniform in size, indicating structural integrity and stable performance of the VLPs. In mice immunized with the hexavalent VLP vaccine, long-lasting and high-titer antibody responses were induced, and specific blocking antibodies and T cell immune responses were generated.

### BRIEF DESCRIPTION OF DRAWINGS

FIG. 1 shows the schematics of plasmid construction.

FIG. 2 shows the results of recombinant plasmid identification by digestion.

FIG. 3 shows the results of Western blot for the selection of dominant strains for each protein.

FIG. 4 shows the results of SDS-PAGE and Western blot for the purification of VLPs.

FIG. 5 shows the transmission electron micrographs of purified VLPs.

FIG. 6 shows the flow chart of immunization with the hexavalent protein.

FIG. 7A-7F show the titers of IgG/IgG1/IgG2a/IgG2b antibodies in immune sera against each of the VLPs: G1.1, G2.2, G2.3, G2.4, G2.6, and G2.17, which are also known as GI.1,GII.2, GII.3, GII.4, GII.6, and GII.17, respectively.

FIG. 8 shows the in vitro blocking test results of antibodies.

FIG. 9 shows the ELISPOT assay results of IFN-γ, IL-2, and TNF-α secretion in spleen cells of immunized mice.

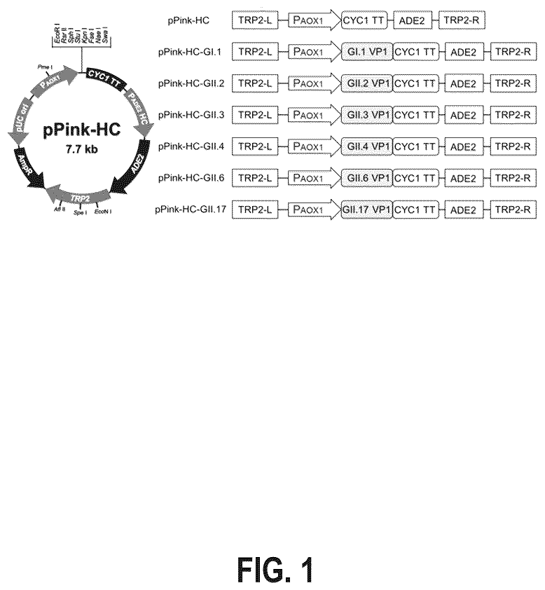

### DETAILED DESCRIPTION

Unless otherwise stated, the methods used in the following examples are conventional methods. Unless otherwise stated, the materials used in the following examples are purchased from commercial sources.

### 1. Construction of Recombinant Vectors

The gene sequences encoding VPI of hexavalent norovirus genes GI.1, GII.3, GII.4, GII.17 (Wang, et al. Viruses, 2018, 10:27; Wang, et al. Vaccine, 2015, 33:57799), GII.2 (VP1 GeneID: 38168211), and GII.6 (VP1 GenBank: KY407216.1) were synthesized by Tsingke Biotechnology Co., Ltd. after yeast genetic codon optimization. The target gene was subcloned into the vector pPink-hc (Invitrogen) of a PichiaPink™ expression system after digestion with the restriction enzymes EcoRI and KpnI.

The VPI sequences of the six norovirus subtypes (optimized nucleotide and amino acid sequences) are as follows: the DNA sequence and the amino acid sequence encoding the protein are respectively shown in SEQ ID No. 1 and SEQ ID No. 2 for GI.1, SEQ ID No.3 and SEQ ID No.4 for GII.2, SEQ ID No. 5 and SEQ ID No. 6 for GII.3, SEQ ID No. 7 and SEQ ID No. 8 for GII.4, SEQ ID No. 9 and SEQ ID No. 10 for GII.6, and SEQ ID No. 11 and SEQ ID No. 12 for GII.17.

### 2. Recombinant Vector Identification

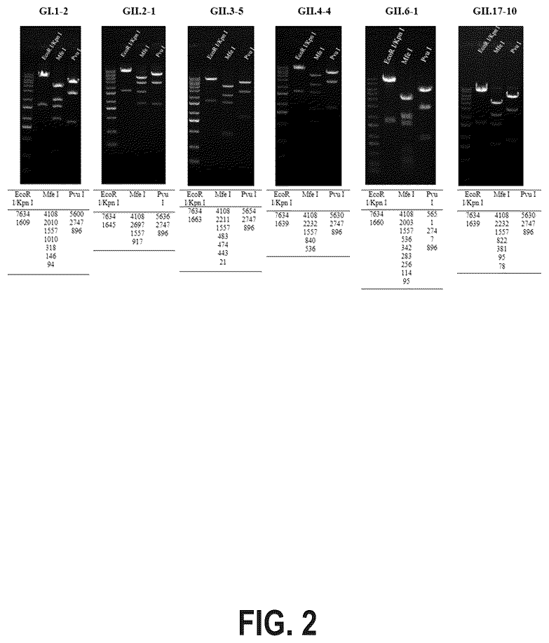

DNA sequencing was performed on the target gene part in the recombinant vectors. The universal primers 5′AOX and CYC1-R were used for sequencing to verify the sequences. The recombinant vectors were subjected to double digestion with EcoRI and KpnI, digestion with Mfe I, and digestion with Pvu I, respectively, to ensure the correct plasmid frame. The results showed that the DNA sequences were correct and the digestion bands were verified. FIG. 2.

The verified correct recombinant plasmids were re-transformed and the transformed cells were spread on LB plates. More than 5 clones were picked after culture to be subjected to overnight culture, and then the plasmids were extracted and subjected to nucleic acid electrophoresis and quantification. Thereby, the plasmids with high quantification were selected for subsequent experiments, which were pPink-hc-GI.1-2, pPink-hc-GII.2-1, pPink-hc-GII.3-5, pPink-hc-GII.4-4, pPink-hc-GII.6-1, and pPink-hc-GII.17-10. The pPink-hc vector was purchased from Thermo Fisher, Waltham, MA, Product no. A11152.

### 3. Amplification and Linearization of Recombinant Vectors

The glycerol stocks of pPink-hc-GI.1-2, pPink-hc-GII.2-1, pPink-hc-GII.3-5, pPink-hc-GII.4-4, pPink-hc-GII.6-1, and pPink-hc-GII.17-10 were separately added into 300 mL of LB medium with 0.1% AMP and cultured at 30° C. and 220 rpm for 12-16 h. Plasmid DNA was purified in large quantities. Linear DNA was recovered after linearization with the restriction enzyme Afl II. The linearized plasmids were transformed into competent yeast strains via electroporation.

### 4. Screening and Expression Identification of Positive Clones

White colonies were picked from PAD plates and rescreened on a new PAD selection plate.

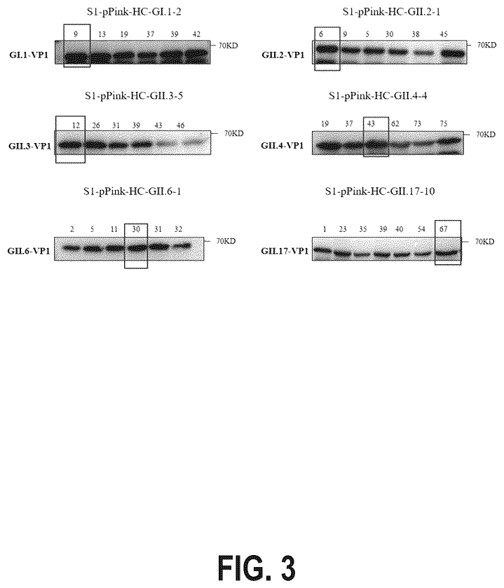

More than 30 positive clones were selected for each genotype. After 48hours of low-level induction, samples from the strains identified as positive by PCR were collected and processed, and the expression of the target protein in these samples was detected by Western blot. Six or seven selected strains of the six genotypes were transferred to 15 mL of BMGY, cultured for 12-16 hours, and centrifuged at 1500 g for 5 min to collect the cells. The cells were resuspended in BMMY to start the induced expression for 5 days. The expression level of each strain was identified by Western blot, and the preferred strains were determined as candidate vaccine strains. The results are shown in FIG. 3. The preferred strains (the candidate vaccine strains) are indicated by the boxes. The dominant yeast strains for each protein were verified by sequencing, which shows that the target genes had been successfully inserted into the yeast genome.

### 5. Purification and Preparation of VLPs

### 5.1 Fermentation in Shaker Flasks at the Laboratory Stage

Six strains, which were S1-pPink-HC-GI.1-2-9, S1-pPink-HC-GII.2-1-6, S1-pPink-HC-GII.3-5-12, S1-pPink-HC-GII.4-4-43, S1-pPink-HC-GII.6-1-30, and S1-pPink-HC-GII.17-10-67, were selected and streaked onto PAD plates to be incubated at 28° C. for 2-3 days. The white clones on the plate were picked to be incubated in 5 mL of BMGY at 28° C. and 220 rpm for 1-2 days (about 30 hours), and then 1-2 mL of the culture was transferred into 500 mL of BMGY and cultured at 28° C. and 220 rpm, with methanol inducer added every 24 hours. After induced expression for 3-5 days, centrifugation was conducted at 5000 g for 5 min to collect the cells, and the cells were washed with ultrapure water 2-3 times.

### 5.2. Purification of VLPs by Sucrose Density Gradient Centrifugation

A 50% sucrose solution was prepared, sterilized at 121° C. for 20 min, and diluted into 40%, 30%, 20%, and 10% sucrose gradient solutions, respectively, for later use.

The cells were suspended in PBS at a concentration of 0.15 M to prepare 10% suspension. After treatment, the supernatant was spread on 10%-50% sucrose gradient solutions and centrifuged at 4° C. and 39,000 rpm for 3 h. 1 mL of the fluid was collected sequentially from top to bottom, and 10 μL of the sample was taken to identify the presence of the target protein with Wester Blots (WB) or SDS-PAGE. The fractions containing the target proteins were recovered and exchanged into 10 mM PBS using 100 KD ultrafiltration tubes. SDS-PAGE was used to detect its purity, WB was used to identify the target protein, and BCA was used to determine the protein concentration.

### 5.3. Purification Results

The purified GI.1, GII.2, GII.3, GII.4, GII.6, and GII.17 proteins obtained from the sucrose gradient purification were exchanged into PBS buffer using ultrafiltration tubes. The protein concentrations were determined using a BCA quantification kit.

### 5.4. Transmission Electron Microscopy

20 The purified VLPs were negatively stained with 2% phosphotungstic acid. The assembly of virus-like particles and particle integrity were observed under a Hitachi HT7700 transmission electron microscope at 80 KV.

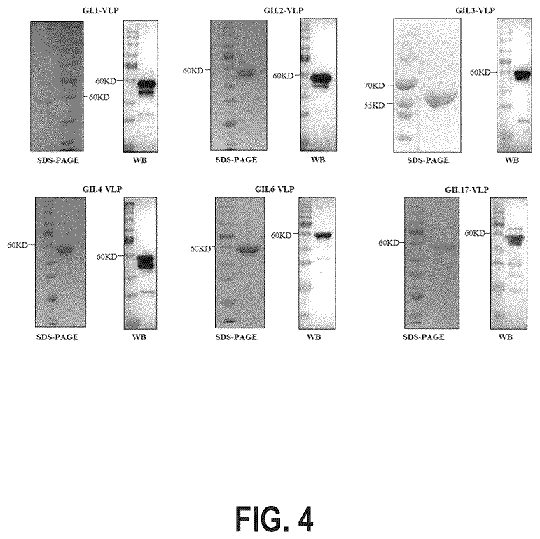

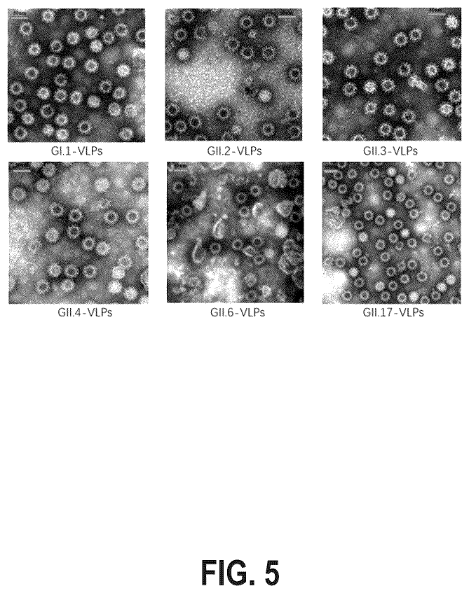

After sucrose density gradient centrifugation, the purified VLP for each protein was obtained, and the purity of each VLP could be over 95% (FIG. 4). The VLP concentrations of GI.1-VLPs, GII.2-VLPs, GII.3-VLPs, GII.4-VLPs, GII.6-VLPs, and GII.17-VLPs were 0.9 mg/mL, 0.6 mg/mL, 1.54 mg/mL, 0.99 mg/mL, 2.1 mg/mL, and 0.8 mg/mL, respectively. Transmission electron microscopy showed that the particles of the VLP for each protein were uniform and structurally intact (FIG. 5).

### 6. Immunization Experiment of the Hexavalent Norovirus VLP Vaccine

### 6.1 Vaccine Preparation

The six proteins were mixed with the aluminum adjuvant (InvivoGen, San Diego, CA) at a volume ratio of 1:1 according to the doses in Table 1, and mixed well by shaking on a shaker at 4° C. for 30 min before immunization of mice.

TABLE 1
Animal grouping and doses

| Group | Quantity | Dose |
|---|---|---|
| 1 | 5 | PBS + Al (OH)3 |
| 2 | 5 | 5 μg GI.1 + Al (OH)3 |
| 3 | 5 | 5 μg GII.2 + Al (OH)3 |
| 4 | 5 | 5 μg GII.3 + Al (OH)3 |
| 5 | 5 | 5 μg GII.4 + Al (OH)3 |
| 6 | 5 | 5 μg GII.6 + Al (OH)3 |
| 7 | 5 | 5 μg GII.17 + Al (OH)3 |
| 8 | 5 | 30 μg (GI.1 + GII.2 + GII.3 + GII.4 + |
| | | GII.6 + GII.17) + Al (OH)3 |

### 6.2. Immunization of Mice

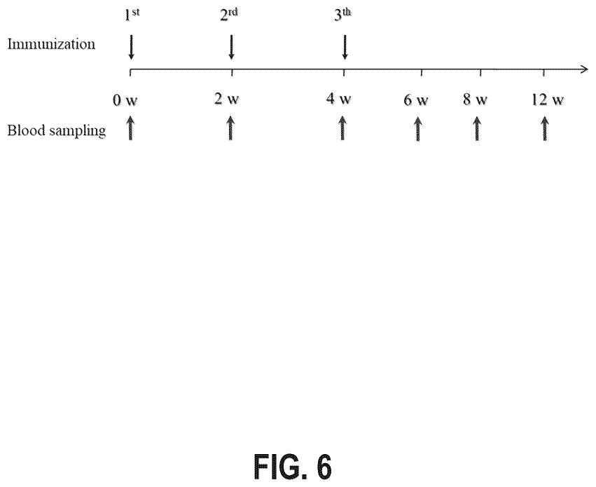

6-to 8-week-old female BALB/c mice (SPF) were randomly divided into 8 groups according to Table 1, with 5 mice in each group. Both hind limbs of the mice were intramuscularly injected with 50 μL each, i.e., a total of 100 μL was injected into each mouse. The immunization and blood sampling procedures were as shown in FIG. 6. The immunization was performed on days 0, 14, and 28, three times in total. Blood samples were collected from the orbit 2 days before each immunization and on Day 42, Day 56, and Day 84. The blood samples were placed in a constant-temperature incubator at 37° C. for 15 min and in a refrigerator at 4° C. for 2 h. The serum was separated by centrifugation at 5000 rpm, 4° C. for 10 min, and at 12,000 rpm, 4° C. for 20 min. The serum was stored in a refrigerator at −80° C.

6.3. Determination of titers of specific antibody IgG\gG1\gG2a\IgG2b in immune sera by indirect ELISA. The titers of IgG, IgG1, IgG2a, and IgG2b antibodies in the sera (previously stored at −80° C.) against each of the VLPs G1.1, G2.2, G2.3, G2.4, G2.6, and G2.17 were detected by indirect ELISA. The procedures were as follows: the microtiter plate was coated with the VLP proteins at 400 ng/100 μL/well, and stored in the refrigerator at 4° C. overnight; the positive serum of each protein and corresponding negative serum at day −2 were taken and diluted by 2-fold serial dilution starting from 1:400, with a total of 12 dilutions; the other sera to be tested were diluted by a factor of 1:400. The microtiter plate was blocked at a constant temperature of 37° C. for 1 h. After the plate was washed, the sera of mice to be tested were added to dilute the samples, 100 μL/well, and two blank control wells were set, and then incubated at 37° C. for 2 h. An antibody diluent was used to dilute Goat Anti-Mouse IgG (HRP) (1:50000), Goat Anti-Mouse IgG1 (HRP) (1:5000), Goat Anti-Mouse IgG2a (HRP) (1:5000), and Goat Anti-Mouse IgG2b (HRP) (1:5000) antibodies, 100 μL/well, at 37° C. for 1 h; after the plate was washed, TMB developing solution was added at 100 L/well to develop color for 5 min; 2M H₂SO₄ was added at 50 μL/well to terminate the reaction. The OD values were read at 450 nm on a microplate reader.

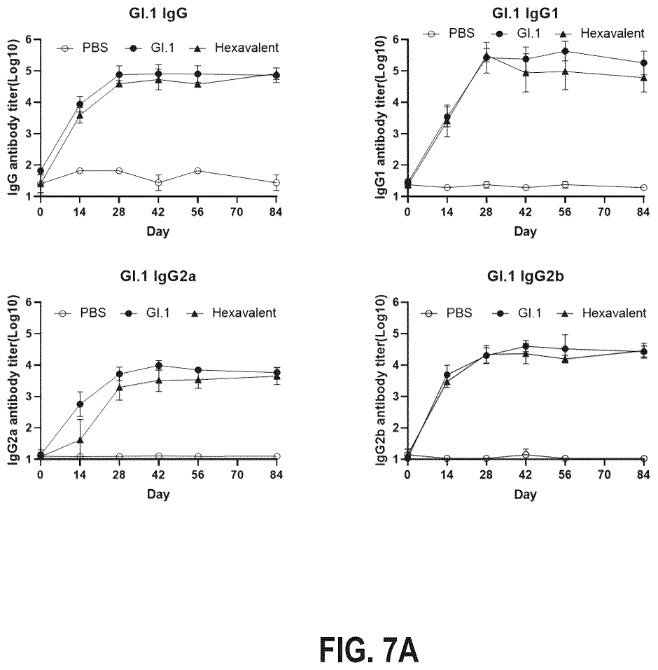

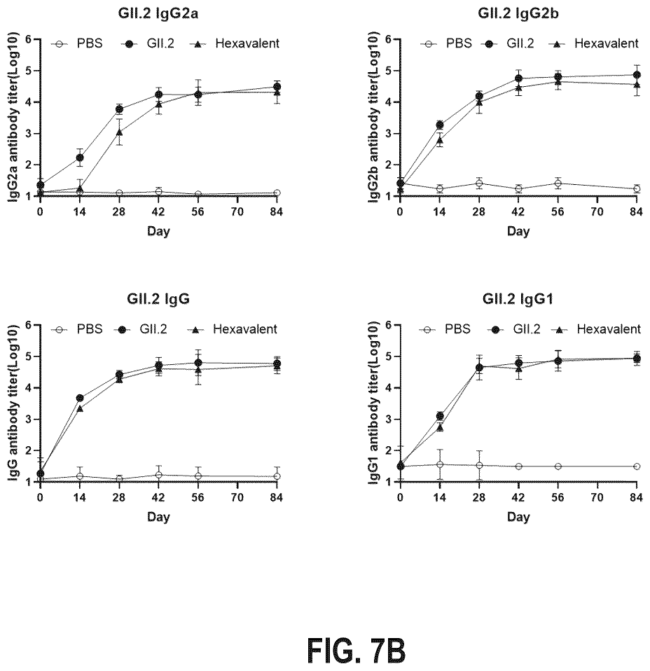

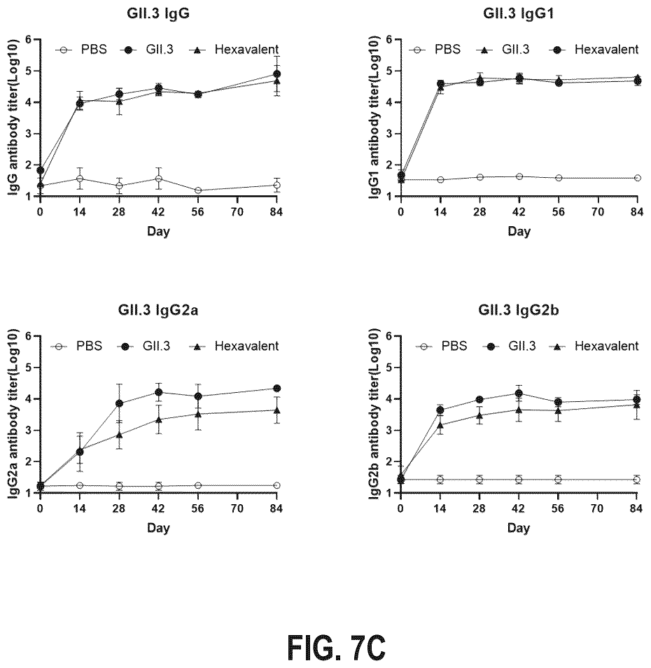

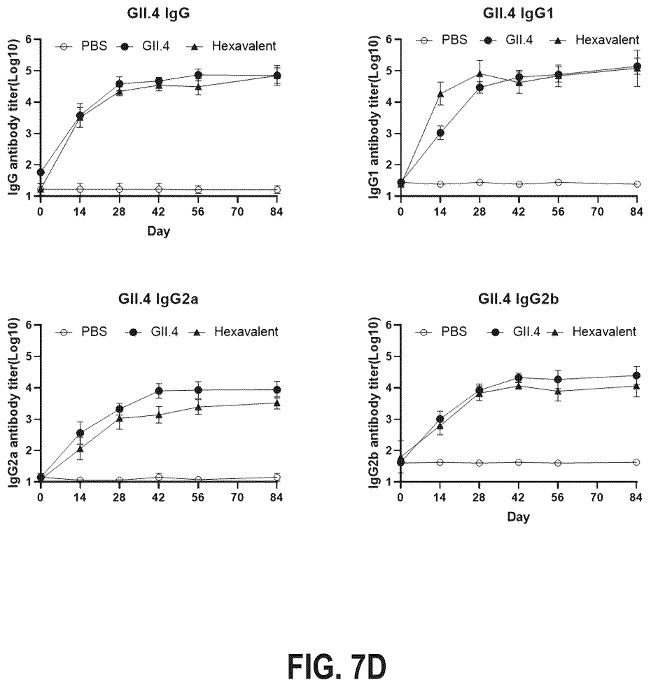

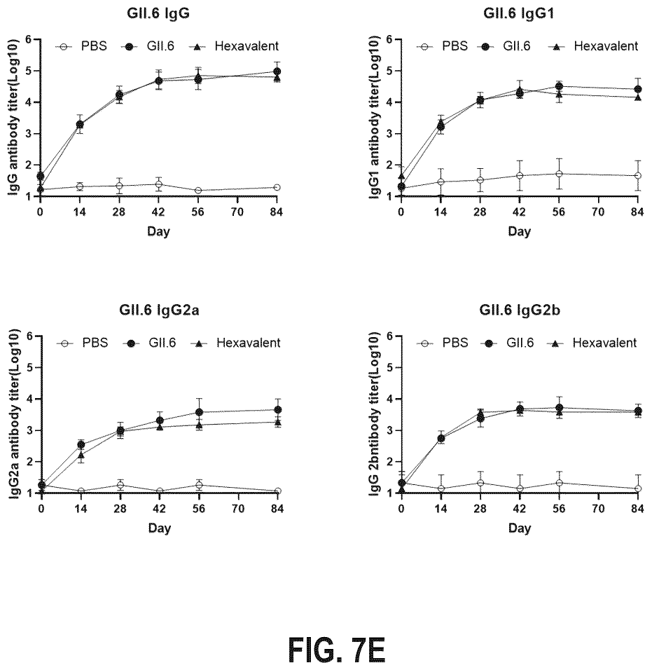

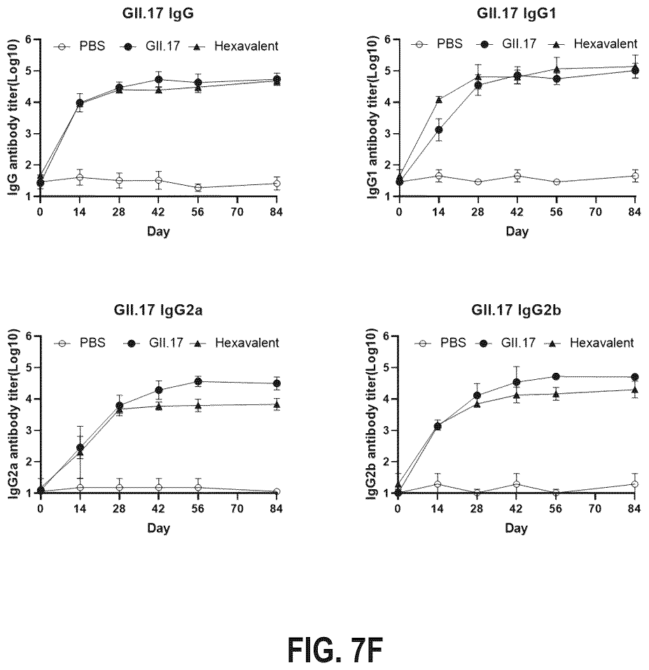

The results of serum IgG detection showed that the antibody levels of each group began to increase on Day 14 after the initial immunization and were significantly higher than those of the PBS group (P<0.001). After the second immunization, the antibody level of each group further increased, and the antibody level continued to decrease significantly until 84 days, with no significant difference between the monovalent and hexavalent groups. In monovalent groups, mice were immunization with a single VLP, for example, GI.1, In hexavalent groups, mice were immunized with all six VLPs: GI.1, GII.2, GII.3, GII.4, GII.6, and GII.17. The results of serum antibody subtype detection (FIGS. 7A-7F) showed that the overall trend of antibody subtypes IgG1, IgG2a, and IgG2b was the same as that of IgG antibodies, and the antibodies did not decrease on Day 84. The results showed that the combination of the hexavalent VLP protein and the aluminum adjuvant could induce Th1 and Th2 immune responses in mice.

### 6.4. In Vitro Blocking Test

The ELISA plate was coated with 20 μg/mL mucin at 50 μL/well at 4° C. overnight, and blocked with 5% skimmed milk at room temperature for 1 h. The 42-day mouse serum was diluted by 2-fold serial dilution with PBST, starting from 1:100, mixed with an equal volume of each VLP, incubated at room temperature for 1 h, and then added to the microtiter plate to be incubated at room temperature for 1 h. Rabbit polyclonal antibody (1:2000) was added and incubated at room temperature for 1 h, HRP-labeled goat anti-rabbit IgG secondary antibody (1:5000) was incubated at room temperature for 1 h, and then 100 μL of TMB was added for color development for 5 min. 50 μL of 2 M H₂SO₄ was added to terminate the reaction. The OD₄₅₀ values were read on a microplate reader. 50% blocking titer (BT50) was defined as the maximum serum dilution that blocked 50% of VLPs from binding to the receptors.

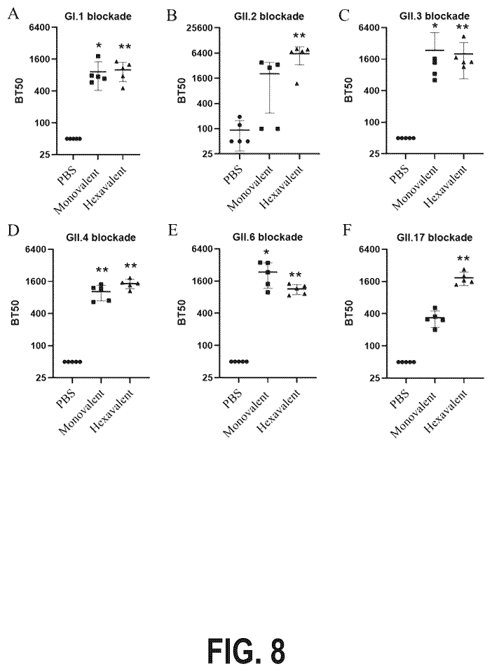

Blocking antibodies are directly related to the protection capability of the vaccine against norovirus infection and pathogenicity. It was demonstrated that immunization with the hexavalent vaccine can induce the same level of blocking antibodies compared to the monovalent vaccine, indicating that the hexavalent vaccine may produce effective immune protection against these epidemic strains after immunization (FIG. 8).

### 6.5. ELISPOT Assays for Secretion Levels of Specific IFN-γ/IL-2/TNF-α

To further confirm the cellular immune effect of hexavalent VLPs, the secretion levels of IFN-γ, IL-2, and TNF-α were determined by ELISPOT:

About 5 mm³ of animal spleen tissues were aseptically taken and put into a plate containing 5 mL of cold IMDM. The tissue was homogenized with ground glass pieces, and the cells on the ground glass pieces were collected into the plate by washing with a buffer using a dropper. The tissue fiber components were removed by filtration with 200-mesh nylon mesh and the spleen cell suspension was transferred into a 10 mL tube and centrifuged horizontally at 1500 rpm for 5 min. The supernatant was discarded, and the residues were mixed well by flipping. 1 mL of red blood cell lysate (0.155 mol/L NH4Cl, 0.01 mol/L KHCO3, and 0.001 mol/L EDTA, pH 7.4) was added, gently mixed with a pipette, and allowed to stand at room temperature for 1 min. IMDM was added to a final volume of 5 mL, and centrifuged horizontally at 1500 rpm for 5 min. The supernatant was discarded, and the residues were mixed well by flipping. Then 2 mL of IMDM was added, and gently mixed with a pipette, and part of the cells were taken for counting. The cells were horizontally centrifuged at 1500 rpm for 5 min, the supernatant was discarded, and the residues were mixed well by flipping; 2 mL of 10% FBS IMDM was added and gently mixed with a pipette for later use.

IFN-γ, IL-2, and TNF-α production were measured according to manufacturer's protocols, i.e., MABTECH AB Mouse IFN-γ ELISPOTBASIC kit, Mouse IL-2 ELISPOTBASIC kit, and TNF-α ELISPOTBASIC kit. Briefly the following procedures were performed:
(1) Antibody coating: The Mouse IFN-γ ELISPOT capture antibody or Mouse IL-2 ELISPOT capture antibody or Mouse TNF-α ELISPOT capture antibody (15 μg/mL) was diluted with 1×PBS and added to the ELISPOT PVDF plate at 100 μL per well, and incubated overnight at 4° C. for coating;
(2) Blocking: The coating solution was discarded, the wells were washed once with a blocking buffer (10% FBS-IMDM), and then the blocking buffer was added at 200 L/well. The plate was incubated at room temperature for 30 min;
(3) Spleen cell activation: The blocking buffer was discarded, the cell suspension was added at 200 μL/well (the number of cells was 6×105 cells/well), and a stimulator was added at 50 μL/well. The final protein concentration was 10 μg/mL. A negative control (spleen cells were stimulated with serum-free medium) and a positive control cell stimulation cocktail (Thermo Scientific) diluted 1:500 were set and incubated in an incubator at 37° C., 5% CO2 for 48 h;
(4) Antibody detection: The cell suspension was discarded and the plate was washed 3 times (3min/time) with deionized water and 4 times with 1×PBST (3 min/time) and finally patted dry on absorbent paper, followed by addition of Mouse IFN-γ ELISPOT detection antibody or Mouse IL-2 ELISPOT detection antibody or Mouse TNF-a ELISPOT detection antibody (1 g/mL) diluted in 10% FBS-PBS at 100 μL/well and incubation at room temperature for 2 h;
(5) Enzyme-labeled antibody: The test antibody was discarded, the plate was washed 5 times with 1×PBST (3 min/time) and patted dry on absorbent paper, and then Streptavidin-HRP diluted in 10% FBS-PBS (1:1000) was added, and incubated for 1 h at room temperature;
(6) Color development: The enzyme-labeled antibody was discarded, and the plate was washed 5 times (3 min/time) with 1×PBST and 4 times (3 min/time) with 1×PBS and then patted dry on absorbent paper. TMB substrate was added at 100 μL/well for color development. Spot formation was observed with naked eyes. Finally, ddH2O was added to terminate the reactions;
(7) Result determination: The ELISPOT plate was dried at room temperature and the number of spots was read using an ELISPOT reader. When the number of spots in control wells was ≤5 spot forming cell (SFC), and that in sample wells ≥10 SFC, it was determined as positive; when the number of spots in control wells was ≥5 SFC but ≤10 SFC, and the number of spots in sample wells/those in control wells >2, it was determined as positive; when the number of spots in control wells was >10 SFC, and the number of spots in sample wells/those in control wells ≥3, it was determined as positive.

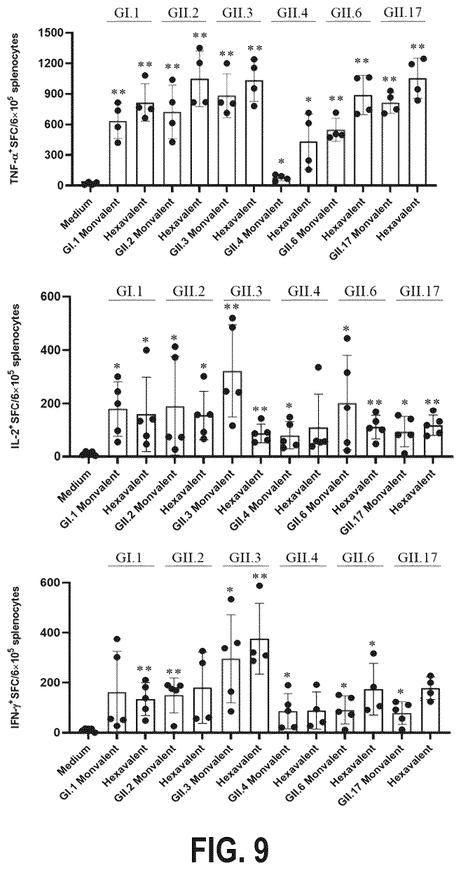

The results are shown in FIG. 9. Both hexavalent and monovalent VLPs were effective in stimulating specific T cell immune responses.

In the present invention, the inventors have successfully obtained correct clones and high-expression vaccine strains by utilizing a Pichia pastoris expression system for hexavalent norovirus vaccine development, and sequencing, digestion, and exogenous gene expression detection for recombinant vector identification and high-expression strain selection. Through VLP purification by sucrose density gradient centrifugation and identification by electron microscopy, the inventors have obtained candidate hexavalent vaccine VLPs that exhibit a purity of over 95%, with each VLP intact in structure and uniform in size, indicating structural integrity and stable performance of the VLPs. In mice immunized with the hexavalent VLP vaccine, long-lasting and high-titer antibody responses were induced, and specific blocking antibodies and T cell immune responses were generated.

In summary, the inventors have successfully developed a hexavalent norovirus VLPP vaccine, which is the world's first hexavalent norovirus VLP vaccine and can be used to treat patient infected with norovirus.

1. A hexavalent norovirus VLP vaccine formulation comprising six antigenic proteins having amino acid sequences shown in SEQ ID No. 2, SEQ ID No. 4, SEQ ID No. 6, SEQ ID No. 8, SEQ ID No. 10, and SEQ ID No. 12.

2. The hexavalent norovirus VLP vaccine formulation of claim 1, wherein the weight ratio of the six antigenic proteins is 1:1:1:1:1:1.

3. The hexavalent norovirus VLP vaccine formulation according to claim 1, further comprising an aluminum adjuvant.

4. The hexavalent norovirus VLP vaccine formulation of claim 3, wherein the aluminum adjuvant is aluminum hydroxide

5. A method for preparing a hexavalent norovirus VLP vaccine formulation, the method comprising:
(1) introducing each of the nucleotide sequences shown in SEQ ID No. 1, SEQ ID No. 3, SEQ ID No. 5, SEQ ID No. 7, SEQ ID No. 9, and SEQ ID No. 11 into a pPink-hc vector, thereby producing six recombinant vectors;
(2) introducing the six recombinant vectors into Pichia pastoris for expression, and collecting and purifying six antigenic proteins encoded by SEQ ID No. 1, SEQ ID No. 3, SEQ ID No. 5, SEQ ID No. 7, SEQ ID No. 9, and SEQ ID No. 11;
(3) mixing the six antigenic proteins purified in step (2) in proportion and encapsulating the mixture with the aluminum adjuvant to obtain the hexavalent norovirus VLP vaccine.

6. The method of claim 5, wherein the weight ratio of the six antigenic proteins is 1:1:1:1:1:1.

7. The method of claim 5, wherein the aluminum adjuvant is aluminum hydroxide.

8. A kit for preparing a hexavalent norovirus VLP vaccine formulation, wherein the kit comprises polynucleotides comprising nucleotide sequences shown in SEQ ID No. 1, SEQ ID No. 3, SEQ ID No. 5, SEQ ID No. 7, SEQ ID No. 9, and SEQ ID No. 11, and a pPink-hc vector.

9. The kit of claim 8, wherein the kit further comprises an aluminum adjuvant.

10. The kit of claim 9, wherein the aluminum adjuvant is aluminum hydroxide.
